(19) United States
(12) Patent Application Publication
(10) Pub. No.: US 2009/0076553 A1
(43) Pub. Date: Mar. 19, 2009

(54) FIXATION SYSTEM FOR BONES

(76) Inventor: Dietmar Wolter, Hamburg (DE)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 12/123,722

(22) Filed: May 20, 2008

Related U.S. Application Data
(63) Continuation-in-part of application No. 10/070,862, filed on Jul. 8, 2002, now abandoned.

(51) Int. Cl. A61B 17/70; A61B 17/04

(52) U.S. Cl. 606/280; 606/286; 606/301; 606/70

(57) ABSTRACT

The invention takes the form of a fixation system for bones with a force carrier (1) having holes (2, 3, 4), two of which are adjacent, and bone screws (36 to 41) adapted to be inserted into the holes (2, 3, 4) the heads of the bone screws (36 to 41) and the holes (2, 3, 4) being adapted to fixedly anchor the heads in the holes (2, 3, 4) wherein the two adjacent holes (2) are disposed on different sides of a zone of fracture or instability of a bone, wherein the force carrier (1) has reinforcements (5) at the holes (2) which are at the smallest distance from the zone of fracture or instability of bone and/or the bone screws (36 to 41) adapted to be inserted into said holes (2) have reinforcements, said reinforcements being dimensioned such that they are able to take up 50 to 60 percent of the total load on the fixation system, wherein the force carrier (1) have further reinforcements (6) at the holes (3) adjacent to the holes (2) mentioned first and/or the bone screws (36 to 41) adapted to be inserted into said holes (3) adjacent to the holes (2) mentioned first have further reinforcements, said further reinforcements (6) being dimensioned such that they are able to take up 20 to 30 percent of the total load on the fixation system and the force carrier (1) is provided with holes (4) which are required to be disposed at an even larger distance from the zone of fracture or instability of a bone, the last mentioned holes (4) and/or the bone screws (36 to 41) adapted to be inserted therein being dimensioned such that the last mentioned holes (4) and/or bone screws (36 to 41) are able to take up to rest of the total load on the fixation system.

### CROSS-REFERENCE TO RELATED APPLICATIONS

This application is a continuation-in-part of Ser. No. 10/070,862, which was a national stage entry of PCT/EP00/08999, with an international filing date of Sep. 14, 2000, the entire contents of each of which is hereby incorporated by reference in their entirety.

### STATEMENT REGARDING FEDERALLY SPONSORED RESEARCH

Not Applicable

### BACKGROUND OF THE INVENTION

The invention relates to a fixation system for bones with a force support having holes and bone screws which are insertable into the holes and are fixable there.

If fragments of broken bones have to be joined to each other plate, nail and fixateur systems are available nowadays. Stability in plate and nail systems has been reached hitherto by the fact that if osteosynthetic plates were used bone screws firmly pulled these plates up to the bones in order to achieve a stabilization of the fragments of broken bones by means of the contact pressure which the plate exerts on the bone. If a marrow nail is used which is in the marrow space stability will also be increased by transversely inserting screws through the bone and the marrow nail. Although these screws are located in the bone by a thread minor motions are possible because the screw passes through the nail.

A fixation plate for the osteosynthesis of the aforementioned type has been known from U.S. Pat. No. 4,683,878, the entire contents is incorporated herein by reference. The plate has a central portion the width of which is distinctly smaller than is the width of the end portions. The end portions of the fixation plate have a plurality of oblong holes. The thickness of each end portion decreases, starting from the central portion to the outer ends of the fixation plate. The plurality of oblong holes have a vertical distal limitation wall and an inclined proximal limitation wall which inclines towards the vertical limitation wall. Each end portion is of a uniform width and the entire lower side of each end portion may be concavely formed to provide a complementary counter-surface to the broken bone. If the bone screws are employed with a head the lower surface of which is rounded the fixation plate pulls the bone pieces together and keeps the bone pieces under a pressure at the point of fracture once the plate and screws are completely fixed to the bone.

Different technical solutions were successful in fixedly joining the head of the screw to the plate or to establish a fixed connection between the screw and the marrow nail itself. In this respect, reference is made to EP 0 201 024 B1, DE 43 43 117 A1, DE 196 29 011 A1, and the German Patent Application P 198 58 889.5, the entire contents of each of which is incorporated by reference in its entirety.

Hence, it is proper to speak of inner fixateur systems in this new generation of implants because the main feature of the outer fixateurs is angular stability between the screw and the transverse force support.

In clinical application, these fixateur-inteme systems have distinctly exhibited a superiority over conventional plate and nail systems up to now.

However, if the patient loads the implant with too much of his body weight too early, which is contrary to a doctor's advice, the implant might be spoiled by bending or the implant might break.

Furthermore, it has been observed that if bones are soft and are subjected to high bending loads the screws can be torn out of the bone.

### BRIEF SUMMARY OF THE INVENTION

Accordingly, it is the object of the invention to improve the fixation system for bones according to the first mentioned document with regard to its load effects.

The invention relies on the surprising finding that the screw hole which is nearest to the zone of fracture or instability of a bone is subjected to the maximum load and that the cause of failure lies in this area.

If a comparison is made between the mechanics of conventional plate systems lacking angular stability to fixateur interne systems enjoying angular stability a fundamental difference can surprisingly be recognized when under a load.

Even if plate systems are used which lack angular stability we will find plate fractures which, as a rule, extend through a plate hole or lead to loosening phenomena with the screws exiting from the bone. If plate systems are used which enjoy angular stability no change of the screw position will occur with respect to the plate itself or the marrow nail because the head of the screw is fixedly anchored in the plate or the screw is fixedly anchored in the marrow nail. This means that forces which act have no uniform effect on a deformation of the plate or the nail as can be encountered more or less in implants which are not angularly stable, but that these forces cause an intensified load by bending in the area of the screw hole which comes to lie closest to the zone of fracture or instability. Even if the patient behaves reasonably and correctly and, hence, a regular course of healing takes place with conventionally dimensioned, angularly stable implants the fracture at this point following the mistake of an overstress is an indication that the forces acting here lead to a damage. At this stage, a failure of the implant might then occur.

Therefore, the invention provides for a reinforcement of the force support at the hole which requires to be disposed in the vicinity of the zone of fracture of instability of a bone. In the case of holes requiring to be disposed farther away, the force support need not have any reinforcement. However, since we can assume that the force support will be under a higher load not only at the first hole, but a higher load will also have to be absorbed by the succeeding hole the hole that follows next also is preferably provided with a reinforcement the dimensions of which, however, may be distinctly smaller. Thus, the adjoining hole may have a reinforcement reduced by about its half. However, the holes which are farther away may be considered to be non-critical, as a rule, and mostly need no reinforcement for this reason.

The reinforcement of the force support may be obtained in different ways:

1. by an increase in the cross-section of the force support, particularly

1.1 by a thickening of the force support (in the direction of the hole), and/or

1.2 by a widening of the force support (in a cross direction of the hole) which can be arc-shaped towards the outside, for example, and/or

1.3 by a reduction in the cross extension (particularly that of a diameter of the hole, and/or

2. by a stronger material of the force support in the hole area.

In preferred aspects of the fixation system, the bone screws can be inserted into the holes of the force support under different angles and can be fixed in the holes. To this effect, the force support and bone screws can be configured according to the patent applications mentioned at the beginning, particularly according to DE 43 43 117 A1, DE 196 29 011 A1, and P 198 58 889.5, the entire contents of each of which is incorporated by reference in its entirety.

One feature of the fixation system is based on the surprising finding that bone screws are susceptible to being torn out particularly if they are introduced into the bone in parallel with each other. To avoid such exiting from the bone, at least two holes which are not in parallel with each other, but are obliquely inclined towards each other, are drilled into the force support.

One feature of fixation system is based on the surprising finding that bone screws are susceptible to extraction particularly if they are introduced into the bone in parallel with each other. To avoid such exiting from the bone, at least two holes are drilled into the force support which are not in parallel with each other, but are obliquely inclined towards each other. In exchange, at least one hole may be obliquely inclined towards the force support as compared to conventional force supports into which the holes are drilled at an angle of 90° from the force support (or from a central plane or a supporting plane thereof on the bone). Preferably, two or more holes can be disposed in an appropriately inclined fashion towards each other. It is preferred here that holes which require to be disposed on different sides of a zone of fracture or instability of a bone are disposed so as to be inclined towards each other in different directions.

Since the bone, as a rule, has curved surfaces and this is the case particularly in the area close to joints it is necessary that plate systems, in particular, be adapted to such bone curvatures. As a rule, this operation is accomplished by appropriate bending tools during the surgery. It is also possible here to vary the orientation of plate holes in accordance with the conformation. If a distinct bone surface curvature is found, e.g. in the area close to joints, the screw hole if obliquely placed may make it even easier to achieve an optimal screw position in the bone. This can be taken into account from the very beginning in orienting holes in the plate so that a desired oblique orientation of at least two holes is achieved in the plate upon conformation.

Preferably, the bone screws may be adapted to be inserted into the holes of the force support under different angles and to be fixed therein. The force support and the screws can be configured here according to the patent applications mentioned at the beginning, particularly according to DE 43 43 117 A1, DE 196 29 011 A1 or P 198 58 889.5, the entire contents of each of which is incorporated by reference in its entirety. The fact that at least two holes are obliquely inclined towards each other in the force support makes it possible to introduce at least two bone screws into the force support so as to be inclined towards each other from the very beginning without using up the clearance furnished by the fixability under different angles. This significantly improves the possibilities to straddle the screws in the bone by giving them an oblique position.

In particular, the fixation system can be a bone plate, a bone nail or a fixateur.

An optimization of the hole configuration with regard to an increase in cross-section adapted to the flow of forces or a selection of the materials or the obliquely oriented holes allow to avoid a possible fracture of the force support if the patient behaves improperly or an extraction of the implant also in case of an overstress.

### BRIEF DESCRIPTION OF THE SEVERAL VIEWS OF THE DRAWINGS

The invention will now be explained in greater detail with reference to the accompanying drawings of embodiments. In the drawings:

FIG. 1 shows a bone plate with widened portions in a partial top view;

FIG. 2 shows a bone plate with thickened portions in a partial longitudinal section;

FIG. 3 shows a curved bone plate on a tibia bone close to a joint in a partial longitudinal section;

FIG. 4 shows a bone plate with inclined holes at the central region of a tubular bone in a longitudinal section;

FIG. 5 shows a bone plate with holes of different diameters at the central region of a tubular bone in a longitudinal section.

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated.

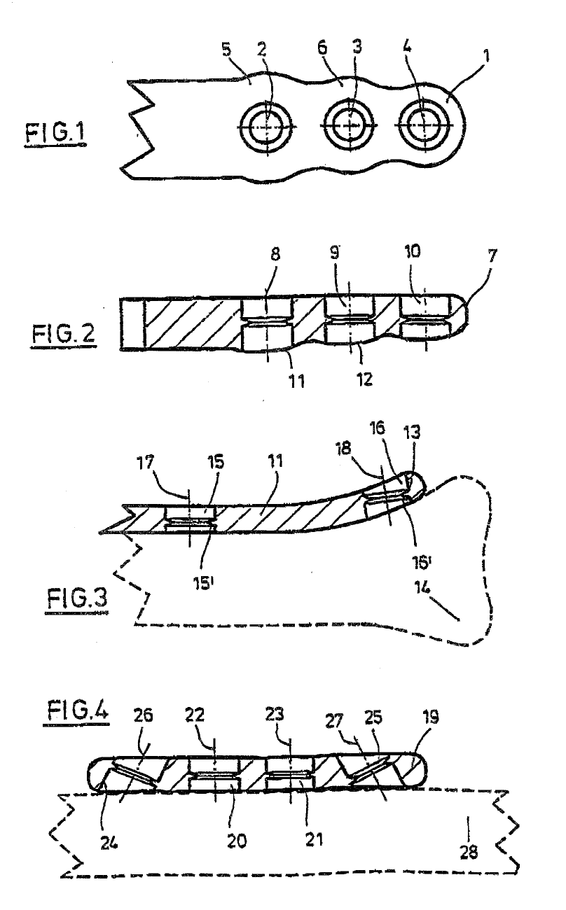

According to FIG. 1, a bone plate 1 has three plate holes 2, 3, 4 in a portion. Of these, plate hole 2 requires to be disposed nearest to a zone of fracture of instability of a bone, hole 3 requires to be disposed at a larger distance, and hole 4 requires to be disposed at the largest distance therefrom. Bone plate 1 has a reinforcement in the form of a large widened portion 5 around hole 2. There is also a reinforcement in the form of a widened portion 6 at hole 3 which, however, is only half the size of the widened portion 5. There is no longer a widened portion at hole 4, but the bone plate is of a substantial constant width here. The widened portions 5, 5 each consist of bent-out regions on the two sides of bone plate 1.

In the cut-away region (on the left in the drawing), which requires to be disposed on the other side of the zone of fracture or instability of a bone, an appropriate sequence of holes may exist with an appropriate reinforcement.

FIG. 2 shows a bone plate 7 the holes 8, 9, 10 of which require to be correspondingly associated with a zone of fracture or instability of a bone. Therefore, it is designed with a maximal thickened portion 11 adjacent to hole 8, a thickened portion 12 which is only half this size adjacent to hole 9, and has no thickened portion adjacent to hole 10.

A bone plate (or another force support) can also have widened portions 5, 6 according to FIG. 1 and thickened portions 11, 12 according to FIG. 2 in a combination.

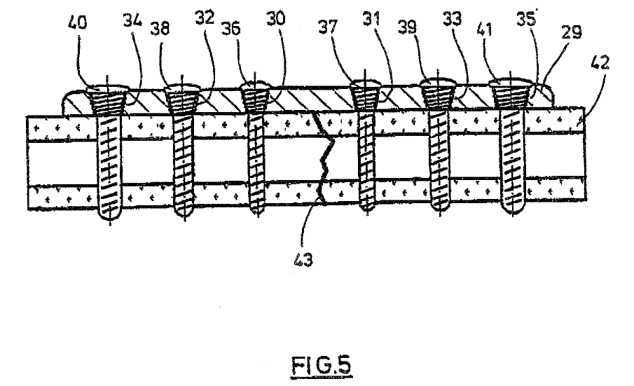

According to FIG. 3, a bone plate 13 has been deformed by bending in such a way that it matches well with the articulation of a tibia bone 14. It has holes 15, 16. More holes may exist on the cut-away part of the bone plate 13. The axis of the bole 15 is oriented perpendicularly to the bone plate 13. The axis of the hole 16 is inclined towards the bone plate or its support surface on the bone from the very beginning. The inclination of the axis of the hole 16 is planned here so as to provide an oblique orientation of the axes of the holes 15, 16 towards each other after the plate is conformed to the bone 14. This causes screws which were turned in to be straddled in the bone, which counteracts an extraction of the implant from the bone.

According to FIG. 3 (and also in all further embodiments), the holes 15, 16 of the bone plate 11 are provided with a circumferential ridge 15′, 16′ at their inner circumference. A bone plate having a thread at the lower side of its head may be turned into this ridge 15′, 16′ in different angular positions, which causes the ridge 15′, 16′ to be reshaped depending on what the angle is from the axis of the hole 15, 16 at which the bone screw is turned in. Moreover, while the ridge or the thread of the screw is being reshaped, the screw is caused to be secured in its turned-in position in the bone. The pre-planned oblique position of the axes of the holes 15, 16 towards each other makes possible an inclined orientation of the bone screws towards each other without using up the clearance furnished for fixability under different angles in the bone plate 11. Thus, a straddled position and optimal orientation of the bone screws in an individually optimizable angular position in their holes 15, 16 are achieved simultaneously.

FIG. 4 shows a bone plate 19 the two middle holes 20, 21 of which have their axes 22, 23 conventionally oriented perpendicularly to the bone plate. The two outer holes 24, 25, however, have their axes 26, 27 oriented at acute angles from the bone plate 18. As a result, the bone screws requiring to be turned into the two outer holes 24, 25 will be straddled in an adjoining bone 28, thus ensuring a safe mounting. This provides a fixability of the bone screws in the holes 20, 21, 24, 25 under different angles also in this example of an application.

FIG. 5 shows a bone plate 29 which has conical holes 30 to 35. These are tapered from the upper side towards the lower side of the bone plate 29.

The two holes 30, 31 which are located nearest to the centre of gravity of the bone plate 29 are of smaller dimensions than are the two holes 32, 33 which are located more outwardly. The latter, in turn, are of smaller dimensions than are the holes 34, 35 which are located most outwardly. Here, the middle holes 30, 31 are of identical dimensions in the example shown. Further, the more outwardly located holes 32, 33 are of coinciding dimensions. Ultimately, the pair of holes 34, 35 also are of coinciding dimensions.

Because of the size distribution depicted for the holes 30, 35, the bone plate 29 has the largest cross-section in the area of holes 30, 31, a slightly smaller cross-section in the area of holes 32, 33, and the smallest cross-section in the area of holes 34, 35. The bone plate 29 can be designed to have a constant thickness and width at any point. As a principle, however, a widened portion and/or thickened portion according to the embodiments of FIGS. 1 and 2 may be added.

By means of bone screws 36 to 41 which were turned in, the bone plate 29 is fixed to a tubular bone 42 in such a way that a fracture zone is disposed precisely between the two central holes 30, 31. The force introduced into the bone plate 29 via the bone screws 36 to 41 is the larger the closer the respective bone screw is located to the fracture zone 43. The bone plate 29 corresponds to this fact, with regard to strength, by being of a cross-section which is the larger the closer the holes 30 to 35 are located to the fracture zone 43.

Incidentally, the bone screws 36 to 41 have a conical thread each, which is turned into the respective hole 30 to 35, above their threaded shanks at the lower side of their heads. Because of their conicity, the threads can be tuned in at different angular orientations. At this stage, the thread digs its way into the inner surface of the respective hole 30 to 35 at the lower side of the head, resulting in the angular position to be fixed as turned in. What is shown in FIG. 5, however, is an orientation of all bone screws 36 to 41 at an angle of 90° from the plane of the bone plate 29.

The invention takes the form of a fixation system for bones with a force carrier (1) having holes (2, 3, 4), two of which are adjacent, and bone screws (36 to 41) adapted to be inserted into the holes (2, 3, 4) the heads of the bone screws (36 to 41) and the holes (2, 3, 4) being adapted to fixedly anchor the heads in the holes (2, 3, 4) wherein the two adjacent holes (2) are disposed on different sides of a zone of fracture or instability of a bone, wherein the force carrier (1) has reinforcements (5) at the holes (2) which are at the smallest distance from the zone of fracture or instability of bone and/or the bone screws (36 to 41) adapted to be inserted into said holes (2) have reinforcements, said reinforcements being dimensioned such that they are able to take up 50 to 60 percent of the total load on the fixation system, wherein the force carrier (1) have further reinforcements (6) at the holes (3) adjacent to the holes (2) mentioned first and/or the bone screws (36 to 41) adapted to be inserted into said holes (3) adjacent to the holes (2) mentioned first have further reinforcements, said further reinforcements (6) being dimensioned such that they are able to take up 20 to 30 percent of the total load on the fixation system and the force carrier (1) is provided with holes (4) which are required to be disposed at an even larger distance from the zone of fracture or instability of a bone, the last mentioned holes (4) and/or the bone screws (36 to 41) adapted to be inserted therein being dimensioned such that the last mentioned holes (4) and/or bone screws (36 to 41) are able to take up to rest of the total load on the fixation system.

Another feature of the invention is that the reinforcements (5, 6) of the force carrier being any one or a combination of the following group consisting of having a widened portion (5, 6) of the force carrier (1) and/or a reduction of the transverse extension of the hole (2, 3, 4) of the force carrier (1) and/or an area where the material of the force carrier (1) is of larger strength.

Another feature of the invention is that the reinforcement of the bone screws (36 to 41) comprises an increased core diameter of a thread on the shank of the screw.

Another feature of the invention is that the reinforcement of the bone screws (36 to 41) comprises a portion of the shank with an increased diameter arranged between the thread on the shank and the head of the screw.

Another feature of the invention is that at least two holes (2, 3, 4) are obliquely inclined towards each other.

Another feature of the invention is that the axis of the two holes (2, 3, 4) diverge on the side of the force carrier (1) which is to face the bone.

Another feature of the invention is that the at least two holes (2, 3, 4) obliquely inclined towards each other are disposed on different sides of the portion of the force carrier (1) which requires to be associated with the zone of fracture or instability of a bone.

Another feature of the invention is that the bone screws (36 to 41) are adapted to be inserted into and fixed in the holes (2, 3, 4) of the force carrier (1) under different angles.

Another feature of the invention is that the bone screws (36 to 41) have a conical thread which is adapted be threaded into the holes (2, 3, 4) in different angular orientations, causing the bone screws (36 to 41) to be fixed in the holes (2, 3, 4).

Another feature of the invention is that the force carrier (1) is a bone plate, a bone nail or a fixateur.

The above Examples and disclosure are intended to be illustrative and not exhaustive. These example and description will suggest many variations and alternatives to one of the ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

What is claimed is:

1. A fixation system for bones with a force carrier (1) having holes (2, 3, 4), two of which are adjacent, and bone screws (36 to 41) adapted to be inserted into the holes (2, 3, 4) the heads of the bone screws (36 to 41) and the holes (2, 3, 4) being adapted to fixedly anchor the heads in the holes (2, 3, 4) wherein the two adjacent holes (2) are disposed on different sides of a zone of fracture or instability of a bone, wherein the force carrier (1) has reinforcements (5) at the holes (2) which are at the smallest distance from the zone of fracture or instability of bone and/or the bone screws (36 to 41) adapted to be inserted into said holes (2) have reinforcements, said reinforcements being dimensioned such that they are able to take up 50 to 60 percent of the total load on the fixation system, wherein the force carrier (1) have further reinforcements (6) at the holes (3) adjacent to the holes (2) mentioned first and/or the bone screws (36 to 41) adapted to be inserted into said holes (3) adjacent to the holes (2) mentioned first have further reinforcements, said further reinforcements (6) being dimensioned such that they are able to take up 20 to 30 percent of the total load on the fixation system and the force carrier (1) is provided with holes (4) which are required to be disposed at an even larger distance from the zone of fracture or instability of a bone, the last mentioned holes (4) and/or the bone screws (36 to 41) adapted to be inserted therein being dimensioned such that the last mentioned holes (4) and/or bone screws (36 to 41) are able to take up to rest of the total load on the fixation system.

2. Fixation system according to claim 1 wherein the reinforcements (5, 6) of the force carrier being any one or a combination of the following group consisting of having a widened portion (5, 6) of the force carrier (1) and/or a reduction of the transverse extension of the hole (2, 3, 4) of the force carrier (1) and/or an area where the material of the force carrier (1) is of larger strength.

3. Fixation system according to claim 1 wherein the reinforcement of the bone screws (36 to 41) comprises an increased core diameter of a thread on the shank of the screw.

4. Fixation system according to claim 1, wherein the reinforcement of the bone screws (36 to 41) comprises a portion of the shank with an increased diameter arranged between the thread on the shank and the head of the screw.

5. Fixation system according to claim 1, wherein at least two holes (2, 3, 4) are obliquely inclined towards each other.

6. Fixation system according to claim 5 wherein the axis of the two holes (2 3, 4) diverge on the side of the force carrier (1) which is to face the bone.

7. Fixation system according to claim 6 wherein the at least two holes (2, 3, 4) obliquely inclined towards each other are disposed on different sides of the portion of the force canier (1) which requires to be associated with the zone of fracture or instability of a bone.

8. Fixation system according to claim 1 wherein the bone screws (36 to 41) are adapted to be inserted into and fixed in the holes (2, 3, 4) of the force carrier (1) under different angles.

9. Fixation system according to claim 8 wherein the bone screws (36 to 41) have a conical thread which is adapted be threaded into the holes (2, 3, 4) in different angular orientations, causing the bone screws (36 to 41) to be fixed in the holes (2, 3, 4).

10. Fixation system according to claim 1, wherein the force carrier (1) is a bone plate, a bone nail or a fixateur.
